(19) United States
(12) Patent Application Publication
(10) Pub. No.: US 2010/0166718 A1
(43) Pub. Date: Jul. 1, 2010

(54) LARGE SCALE PANCREATIC ISLET PURIFICATION

(75) Inventors: Shinichi Matsumoto, Arlington, TX (US); Masayuki Shimoda, Irving, TX (US)

Correspondence Address: CHALKER FLORES, LLP, 2711 LBJ FRWY, Suite 1036, DALLAS, TX, 75234, US

(73) Assignee: Baylor Research Institute, Dallas, TX (US)

(21) Appl. No.: 12/645,910

(22) Filed: Dec. 23, 2009

Related U.S. Application Data
(60) Provisional application No. 61/140,910, filed on Dec. 26, 2008.

(51) Int. Cl. A61K 35/39; C12N 5/07; C12N 5/071; A61P 3/10

(52) U.S. Cl. 424/93.7; 435/325; 435/366

(57) ABSTRACT

The present invention includes a method of isolating pancreatic islets by density centrifugation wherein the pancreatic islets are loaded in a solution comprising pancreatic islets with the density gradient and the islets are isolated by centrifuging the vessel wherein the pressure on the pancreatic islets is less than 50 Pa and wherein the pancreatic islets are isolated from the gradient, the improvement comprising creating a continuous density gradient in a vessel comprising at least 100 milliliters with a bend tube that reduces convection currents that disrupt the gradient.

### TECHNICAL FIELD OF THE INVENTION

The present invention relates to a method of isolating pancreatic islets, which is a critical technology for pancreatic islet transplantation. More particularly, the present invention relates to an apparatus and method for improving the yield of whole pancreatic islets that uses common laboratory equipment and techniques known to the artisan skilled in pancreatic islet isolation.

### STATEMENT OF FEDERALLY FUNDED RESEARCH

None.

### CROSS-REFERENCE TO RELATED APPLICATIONS

This application is related to U.S. Provisional Application Ser. No. 61/140,910, filed Dec. 26, 2008, the entire contents of which are incorporated herein by reference.

### BACKGROUND OF THE INVENTION

Without limiting the scope of the invention, its background is described in connection with methods and devices for pancreatic islet isolation.

The technique of pancreatic islet transplantation into type-1 diabetic patients, who are unable to survive without the administration of insulin, that is, who are in an insulin dependent diabetes mellitus, is garnering a great deal of public awareness and efforts are being made, mainly in Europe and the United States, to establish this technique as a clinical treatment.

Pancreatic islet transplantation refers to cellular tissue transplantation in which pancreatic islet cell groups, which play a central role in blood sugar regulation in the body, are administered by infusion into the portal vein. Islet transplantation is minimally invasive for the transplant recipient and is regarded as the treatment nearest to ideal for type-1 diabetic patients.

In 2000, at the University of Alberta in Edmonton, Canada, a successful trial of clinical islet transplantation was reported. Since this report, approximately 300 islet transplantations have been performed in the 4 years, mainly in Europe and the United States. These islet transplantations have been carried out on the basis of the Edmonton protocol established at the University of Alberta.

However, with current technology, consistent islet yields have not been obtained, even in islet transplantation from brain-dead donors carried out in Europe and the United States, and in some instances the transplanted islets have also not functioned effectively. Moreover, even when considered on a worldwide basis, there have been almost no successful cases of islet transplantation from non-heart-beating donors, where the conditions are worse than with brain-dead donors, and in fact islet transplantation from non-heart-beating donors has to date not been possible.

To improve the success rates of islet transplantation and also to achieve successful islet transplantation from non-heart-beating donors, it is important to transplant a large population of islets fit for transplantation. Therefore, there has been strong demand for improvements in islet isolation technology in order to raise the yield of transplantable islets.

In the medical treatment of transplantation a method was reported in which ulinastatin or a ulinastatin substitute is administered post-transplant to organ transplant patients (See Japanese Unexamined Patent Publication No. 2002-20309).

Furthermore, a solution for perfusion or storage of organs that are destined for transplantation has been reported, that provided valuable results in lung transplantation (see Japanese Unexamined Patent Publication No. H6-40801). However, an optimal means for islet transplantation, particularly with regard to islet isolation and purification technology, remains elusive.

One such apparatus and method is taught in U.S. Pat. No. 7,045,349, issued to Benedict, et al., for a method of islet isolation using process control. Briefly, an “Advanced Islet Separation Technology” is taught that is said to incorporate an automated method, automated control methodology, process control interface, and automated apparatus to separate (isolate) and process pancreatic islets in a tissue suspension in physiologic process solution, utilizing microprocessor controllers and computer control and software programming to interface and control the process temperature, process flowrate, percent hydrogen concentration, dissolved oxygen concentration, endotoxin concentration, dissolved nitric oxide concentration, nitric oxide synthase concentration, proteolytic enzyme activity, and pressure of the islet containing physiologic process solution, including real-time process data acquisition and recording of the process variables.

Yet another invention is taught in United States Patent Application Serial No. 20080038823, filed by Matsumoto, et al., for a method of separating pancreatic islets. Briefly, invention provides a pancreatic islet isolation method comprising the steps of: injecting a protection solution containing a protease inhibitor into the pancreatic duct of a procured pancreas; digesting the pancreas into which the protection solution has been injected; and purifying the digested pancreatic tissue using a purification solution containing a density gradient reagent. The present invention also provides a protection solution for injection into the pancreatic duct, a pancreas preservation solution for the two-layer method, and an islet purification solution.

### SUMMARY OF THE INVENTION

The present invention describes a new purification method using large plastic bottles for the increasing the efficacy of islet purification. The method of the present invention addresses the problem associated with the standard purification method using COBE 2991 cell processor (COBE) with Ficoll density gradient solution which tends to damage islets mechanically by high shearing force.

In one embodiment, the present invention includes an apparatus and method of isolating pancreatic islets comprising: creating a continuous density gradient in a vessel comprising at least 100 milliliters with a bend tube that reduces convection currents that disrupt the gradient; loading a solution comprising pancreatic islets in the density gradient; centrifuging the vessel wherein the pressure on the pancreatic islets is less than 50 Pa; and isolating the pancreatic islets from the gradient. In one aspect, the gradient and the solution comprising pancreatic islets is topped with a dilution solution. In another aspect, the pancreatic islets are top-loaded and in which the gradient comprises: a high-density first layer at the bottom of the vessel, a second layer comprising the continuous density gradient on the high density first layer, a third layer on the second layer, the third layer comprising a low-density solution that comprises a pancreatic digest; and a fourth layer on the third layer comprising a dilution solution. In yet another aspect, the pancreatic islets are bottom-loaded and in which the gradient comprises: a high-density first layer comprising a pancreatic digest at the bottom of the vessel; a second layer on the first layer comprising a continuous density gradient; and a third layer comprising a dilution solution.

In one aspect, layer comprising a dilution solution. In one aspect the gradient comprises a continuous density solution, further defined as comprising a low-density to a high-density by changing the volumetric ratio of low-density to high-density using the bend tube. In another aspect, the gradient comprises a continuous density solution, further defined as comprising a low-density to a high-density comprising Iodixanol to ET-Kyoto solutions produced by changing the volumetric ratio of Iodixanol and ET-Kyoto solution using the bend tube. In yet another aspect, the gradient comprises a continuous density solution, further defined as comprising a low-density (density: 1.077) to a high-density (density: 1.095-1.125) Iodixanol-ET-Kyoto solutions produced by changing the volumetric ratio of Iodixanol and ET-Kyoto solution using the bend tube. In one aspect, the bottle has a smooth inner surface. In one aspect, wherein the gradient is formed using a bent-tip that reduces convection currents in the gradient when the gradient is created. In one aspect, the vessel is 200, 250, 300, 400, 500, 600, 700, 800, 900 or 1,000 milliliters. In yet another aspect, the method further comprises the step of isolating the islet cells from the collection vessel comprising the islet cells. In one aspect, the method further comprises the steps of: isolating the islet cells from the collection vessel comprising the islet cells; washing the islet cells; and transplanting the pancreatic islets into a new host. In one aspect, the islets are human islets. In another aspect, the pancreatic islets are cadaveric islets.

In another embodiment, the present invention includes an apparatus and method of isolating pancreatic islets comprising: creating a density gradient in a vessel comprising at least 100 milliliters; loading a solution comprising pancreatic islets on top of the density gradient; centrifuging the vessel wherein the pressure on the pancreatic islets is less than 50 Pa; separating into two or more collection vessels the gradient from the top to the bottom of the vessel; selecting the collection vessel with the pancreatic islets; and isolating the pancreatic islets from the gradient.

In yet another embodiment, the present invention includes an apparatus and method of isolating pancreatic islets comprising: creating a density gradient in a vessel comprising at least 100 milliliters; loading a solution comprising pancreatic islets below the density gradient; centrifuging the vessel wherein the pressure on the pancreatic islets is less than 50 Pa; separating into two or more collection vessels the gradient from the top to the bottom of the vessel; selecting the collection vessel with the pancreatic islets; and isolating the pancreatic islets from the gradient.

Yet another embodiment, includes an apparatus and method of isolating pancreatic islets by density centrifugation wherein the pancreatic islets are loaded in a solution comprising pancreatic islets with the density gradient and the islets are isolated by centrifuging the vessel wherein the pressure on the pancreatic islets is less than 50 Pa and wherein the pancreatic islets are isolated from the gradient, the improvement comprising creating a continuous density gradient in a vessel comprising at least 100 milliliters with a bend tube that reduces convection currents that disrupt the gradient.

Yet another embodiment of the present invention includes one or more isolated pancreatic cells isolated by a method that comprises: creating a continuous density gradient in a vessel comprising at least 100 milliliters with a bend tube that reduces convection currents that disrupt the gradient; loading a solution comprising pancreatic islets in the density gradient; centrifuging the vessel wherein the pressure on the pancreatic islets is less than 50 Pa; and isolating the pancreatic islets from the gradient. In one aspect, the gradient and the solution comprising pancreatic islets is topped with a dilution solution. In another aspect, the pancreatic islets are top-loaded and in which the gradient comprises: a high-density first layer at the bottom of the vessel, a second layer comprising the continuous density gradient on the high density first layer, a third layer on the second layer, the third layer comprising a low-density solution that comprises a pancreatic digest; and a fourth layer on the third layer comprising a dilution solution. In yet another aspect, the pancreatic islets are bottom-loaded and in which the gradient comprises: a high-density first layer comprising a pancreatic digest at the bottom of the vessel; a second layer on the first layer comprising a continuous density gradient; and a third layer comprising a dilution solution.

### BRIEF DESCRIPTION OF THE DRAWINGS

For a more complete understanding of the features and advantages of the present invention, reference is now made to the detailed description of the invention along with the accompanying figures and in which:

FIGS. 1A to 1D show a morphological assessment of purified islets with different shearing forces. Islets before application of shearing force (A). Shearing force was applied on those islets (B: 100 Pa for 60 sec., C: 100 Pa for 180 sec., and D: 50 Pa for 300 sec.).

FIG. 2A shows the schema of the bottle purification method of the present invention. First the continuous gradient solution including the digested tissue was generated in large bottles with a gradient maker. Then, they were centrifuged at 1000 rpm for 5 minutes at 4° C. and the solution was collected into 10 tubes.

FIG. 2B shows a “Tip-bent” candy-cane shaped stainless pipe used for making the gradient.

FIG. 2C is a picture during making the gradient (Bottom loading).

FIG. 3 shows pictures of the bottles after centrifugation. Left: Top loading. Right: Bottom loading. Islets exist in the upper layer in both methods.

FIGS. 4 and 5 are graphs that show that the continuous density gradient can be maintained in both top and bottom loading methods during the purification.

FIG. 6 shows the steps followed to compare the current art with the present invention.

FIG. 7 are images showing the morphology of Post-purification Islets. The same volume samples after purification were taken from three methods. In the Top method, large islets were seen. Original magnification: ×40, Scale bar: 150 μm

FIG. 8 shows several graphs that compare the results of the prior art (COBE2991) versus the top loading and bottom loading method using bottle purification of Pancreatic Islets for: Post-purification IEQs, IEQ to Islet Particle No.; Recovery rate (%); Purity (%); and Viability (%).

FIG. 9 includes graphs that compare the Pancreatic Islet size for: Average Islet Diameter; Ratio of Islets>200 μm; and Average Islet Size Distribution (%).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Islet transplantation is a promising treatment for Type 1 Diabetes. However, several urgent issues need to be solved. One of such issues is low efficacy of islet isolation and low success rate of islet isolation for transplantation. Even advanced islet transplantation centers achieved less than 50% of successful islet isolation using highest quality of donor pancreata.

Islet isolation process is including donor selection, pancreas procurement and preservation, pancreas digestion and islet purification. Purification process is one of the most difficult procedures for islet isolation. The present inventors have found that using the standard refrigerated COBE 2991 cell processor with human pancreata results in only 53.2±4.5% (mean±SE; Pre-purification/Post-purification islet yield) recovery.

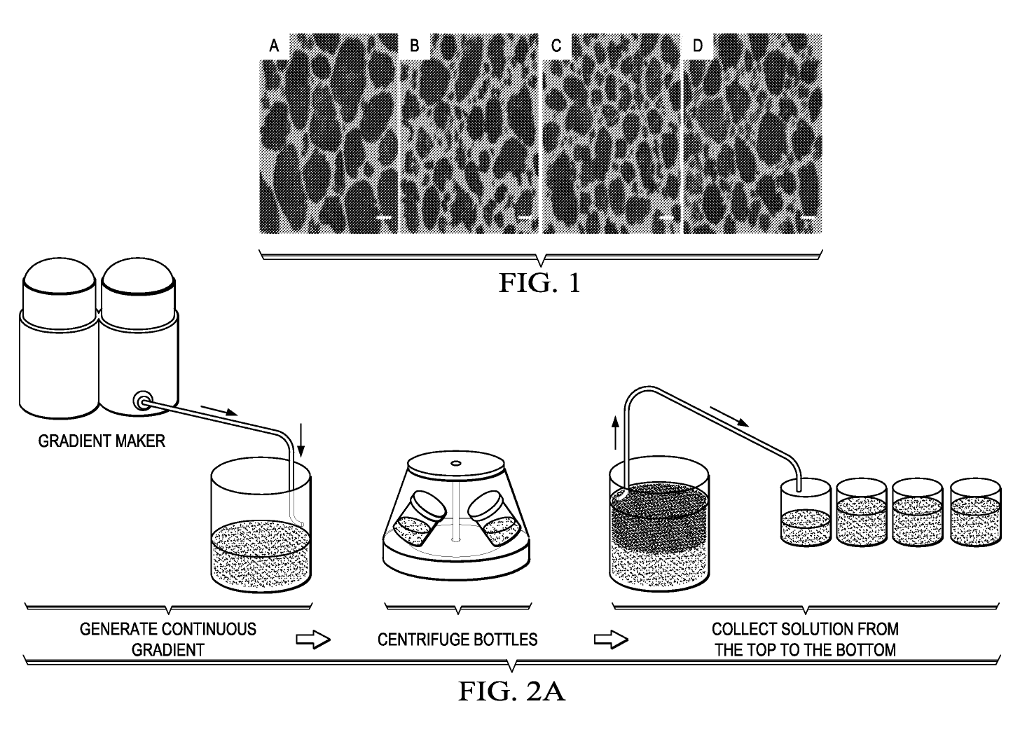

Using the present invention when compared to the standard purification method using a bag cell processor (COBE 2991) with a Ficoll density gradient solution, the cell processor damaged islets mechanically¹. FIG. 1 shows a Morphological assessment of purified islets with different shearing forces. Islets before application of shearing force (A). See, Shintaku H, Okitsu T, Kawano S, Matsumoto S, Suzuki T, Kanno I, Kotera H. Effects of fluid dynamic stress on fracturing of cell-aggregated tissue during purification for islets of Langerhans transplantation. Journal of Physics D: Applied Physics (in press). Shearing force was applied on those islets (B: 100 Pa for 60 sec., C: 100 Pa for 180 sec., and D: 50 Pa for 300 sec.). FIG. 1 demonstrates the profound effect of shearing forces on mechanical damage to islets. FIG. 1 shows that the Islets were falling apart after applying the listed shearing forces.

The present invention includes an apparatus and method for pancreatic islet isolation. The method provides a larger volume of pancreatic islets and does not require the use of a bag cell processor. This method enabled yielded about double the pancreatic islet yield compared with the regular pancreatic islet isolation methods. The method of the present invention uses two large volume (e.g., 100, 200, 250, 300, 400, 500 or 1,000 ml) hard plastic bottles to make density gradient. One advantage of this method is that it avoids shearing stress during islet purification when compared to a cell processor by eliminating its loading and pushing out process. The new method (NM) enabled us to obtain about double the islet yield compared with the regular method (RM) and a much higher yield compared with the open pan method (OPM).

Porcine islet isolation. Porcine pancreata were obtained at a local slaughterhouse. About 30 minutes after the cessation of heart beating, the operation was started. After removing the pancreas, a cannula was immediately inserted into the main pancreatic duct and infused about 200-250 ml of ET-KYOTO solution (Otsuka Pharmaceutical Factory, Japan)²⁻³ with or without Aralast (Baxter, Deerfield, Ill.) for ductal protection, and removed fatty tissues and put the pancreas into the two-layer (ET-KYOTO/PFC) preservation container. Warm ischemic time (WIT) was defined as the time between the cessation of heart beating and the placement of the pancreas into the preservation solution. Cold ischemic time (CIT) was defined as the time between the placement of the pancreas into the preservation solution and the start of islet isolation. Other solutions for use with the present invention include, but are not limited to: ET-Kyoto solution, and the modifications thereto, include trehalose as a nonreducing disaccharide that stabilizes the cell membrane under various stressful conditions. Two variants on ET-Kyoto solution have different electrolyte contents, e.g., Na 100 mmol/L, K 44 mmol/L (so-called “extracellular” solution) and an “intracellular type” IT-Kyoto solution, e.g., Na 20 mmol/L, K 130 mmol/L, with trehalose at 35 gr/l. Non-limiting examples of complete solutions for use with the present invention are summarized in Table 1.

TABLE 1
Preservation Solutions.

| Solution | E-C | C-S | UW | LPD-G | ET-Kyoto | IT-Kyoto | nEt-Kyoto | C |
|---|---|---|---|---|---|---|---|---|
| Na+ | 10 | 17 | 30 | 165 | 100 | 20 | 107 | 100 |
| K+ | 115 | 115 | 125 | 4 | 44 | 130 | 44 | 15 |
| Mg++ | 5 | 5 | 5 | 2 | — | — | — | 13 |
| Ca++ | — | — | — | — | — | — | — | 0.25 |
| Cl— | 15 | 15 | — | 101 | — | — | — | — |
| CO3H— | 10 | 10 | — | — | — | — | — | — |
| PO4H2— | 58 | 58 | 25 | 36 | 26 | 25 | 25 | — |
| SO4═ | 5 | 5 | 5 | — | — | — | — | — |
| Glucose | 195 | — | — | 56 | — | — | — | — |
| Gluconate | — | — | — | — | 100 | 100 | 100 | — |
| Lactobionate | — | — | 100 | — | — | — | — | 80 |
| Adenosine | — | — | 5 | — | — | — | — | 1 |
| Glutamine | — | — | 3 | — | — | — | — | 1 |
| Alopurinol | — | — | 1 | — | — | — | — | 1 |
| Trehalose | — | — | — | — | 120 | — | 120 | — |
| Raffinose | — | — | 30 | — | — | — | — | — |
| Dextran 40(g/L) | — | — | — | 20 | — | — | — | — |
| Mannitol(g/L) | — | 37.5 | — | — | — | — | — | 60 |
| EDTA(g/L) | — | 0.075 | — | — | — | — | — | — |
| HES(g/L) | — | — | 50 | — | 30 | 30 | 30 | — |
| NAC | — | — | — | — | — | — | 10 | — |
| Db c-AMP | — | — | — | — | — | — | 2 | — |
| Nitroglycerine | — | — | — | — | — | — | 0.44 | — |
| pH | 7.4 | 7.4 | 7.4 | 7.4 | 7.4 | 7.4 | 7.4 | 7.3 |
| Osmolarity(**) | 355 | 420 | 325 | 335 | 370 | 370 | 600 | 360 |

E-C: Euro-Collins. C-S: Collins-Sacks. UW: University of Wisconsin - Beltzer. LPD-G: Low potassium Dextran - Glucose. ET-K: Extracellular-type Kyoto. IT-K: Intracellular-type Kyoto. nET-K: new ET-K; C: Celsior. EDTA: ethylenediaminetetraacetic acid. HES: Hydroxyethyl starch. NAC: N-acetylcysteine. Db c-AMP: Dibutyl cyclic AMP. All concentration in mMol/L, except (*)gr/L. (**)Osmolarity is expressed Osm/L.

Examples trypsin inhibitors include, but are not limited to, serum α-1 antitrypsin, a lima bean trypsin inhibitor, a Kunitz inhibitor, a ovomucoid inhibitor or a soybean inhibitor.

Islet isolation was conducted in accordance with the method described in the Edmonton protocol⁴⁻⁸. In brief, after decontamination of the pancreas, the ducts were perfused with 500 ml of Liberase HI (1 mg/ml; Roche Applied Science, Indianapolis, Ind.) solution in a pressure-controlled fashion at 4° C. for 10 minutes. After distention, the whole pancreas was then cut into pieces (e.g., about nine pieces), placed in a sterilized Ricordi dissociation chamber (1 L). Then, the enzyme solution was re-circulated and the temperature was raised up to 37° C. shaking the chamber gently. While the pancreas was being digested, the extent of digestion was monitored with dithizone staining by taking small samples.

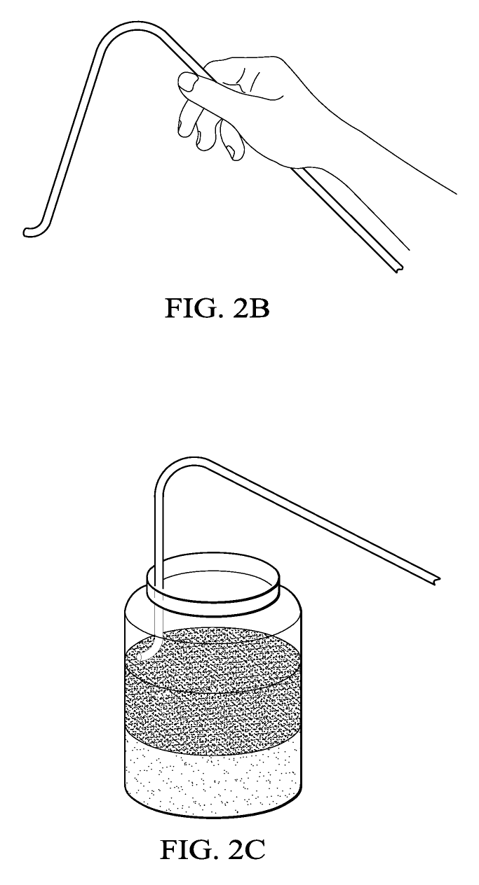

When islets were detected in the sample, we started to collect the solution adding a balances salt solution or other media as a Dilution solution (e.g., UW solution, UW Solution: Potassium lactobionate: 100 mM, KH2PO4: 25 mM, MgSO4: 5 mM, Raffinose: 30 mM, Adenosine: 5 mM, Glutathine: 3 mM, Allopurinol: 1 mM, Hydroxyethyl starch: 50 g/L, adjust pH to 7.4 with KOH, add prior to use: optionally, dexamethasone (8 mg/L), insulin (40 U/L), penicillin (200,000 U/L), final salt values: Na=25+/−5 mM; K=120+/−5 mM; mOsm/L=320+/−10.) or other dilution media such as DMEM/F12 medium from, e.g., Mediatech, Manassas, Va.) into the system for dilution. The digested tissue was collected into Dilution solution that is made of 10% fetal bovine serum and then washed with UW solution (ViaSpan, DuPont Pharmaceuticals, Wilmington, Del.) added with Heparin (5,000 U/L) to remove the enzyme. The Phase I period was defined as the time between the placement of the pancreas in the Ricordi chamber and the start of collecting the digested pancreas. The Phase II period was defined as the time between the start and end of the collection. FIGS. 2A-2C shows a basic outline of the bottle purification method.

Islet Purification. The pancreatic digest was separated into exact halves. One half was purified using COBE 2991 cell processor (COBE), (CaridianBCT, Denver, Colo.)⁹⁻¹¹. The other half was separated into two parts again, and one quarter was purified with the “Top loading” Bottle Method (Top) and the other quarter was done with the “Bottom loading” Bottle Method (Bottom). All these methods were conducted with a continuous density gradient with Iodixanol-ET-Kyoto solution. Since Iodixanol has low viscosity, it needs slower centrifugation for the distribution than Ficoll. For the continuous density solution, low-density (density: 1.077) and high-density (density: 1.095-1.125) Iodixanol-ET-Kyoto solutions were produced by changing the volumetric ratio of Iodixanol and ET-Kyoto solution. The high-density was determined by testing small digest samples centrifuged in each density on the same condition as the performance. For the bottle purification, any size vessel or container can be used, e.g., one or two 100, 200, 250, 300, 400, 500 ml or larger flat bottom plastic containers, one was used for “Top” and the other was used for “Bottom”

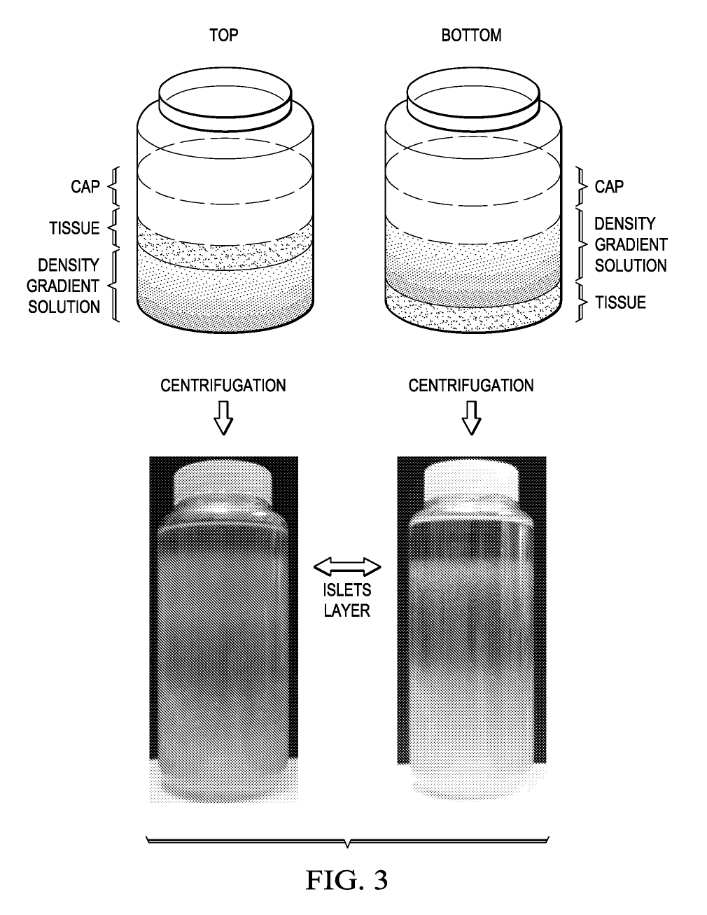

In “Top” method, the gradient consisted 50 ml of high-density at the bottom layer and then 300 ml of continuous density gradient in the 2nd layer and 50 ml of low-density including the pancreatic digest in the 3rd layer and 50 ml of Dilution solution on the top as a cap solution. In “Bottom” method, 100 ml of high-density including the digest was at the bottom and 300 ml of continuous density gradient was in the 2nd layer and 50 ml of cap solution was on the top. The continuous gradients were produced with a gradient maker (FIG. 2A). For the bottle method, a “Bent-Tip” or “candy-cane” shaped stainless pipe (Umihara, Kyoto, Japan) was developed to pour the solution into the bottle to minimize convection currents. The bent-tip minimizes the convection current and maintains the gradient even in the Top-loading methods. The gradients were centrifuged at 1000 rpm for 5 minutes at 4° C. in all systems. After centrifugation, islets were seen in the upper layer of the bottles (FIG. 3).

FIGS. 2B-2C shows the components and steps to load the gradient and the isolated of islets following separation using a “bent-tip” that minimizes the convection current and helps maintain the gradient. The bent-tip comprises a tube that has an about 60, 65, 70, 80, 90, 100, 110, 115 or 120 degree bend that is used to form the gradient and that minimizes convection current while making the gradient (thereby enhancing the consistency of the gradient). By forming the continuous density gradient by adding liquid at an angle generally parallel to the gradient, it has been found that there is an unexpected improvement in islet yield and quality. Furthermore, using the three-part gradient an improvement was also identified.

After centrifugation, the gradient solution was collected into 10 tubes from the top to the bottom (FIG. 2A). To confirm stability of continuous density gradient, we measured density of each 10 fractions before applying this method for islet purification. In COBE method, about 80 ml of the solution was left in the COBE bag structurally. On the other hand, whole solution was collected in the bottle method, which can prevent the loss of islets. Samples were taken from each tube, stained with dithizone (Sigma Chemical Co., St. Louis, Mo.) and assessed for islet purity. Islet-rich tubes were selected and the tissues were collected.

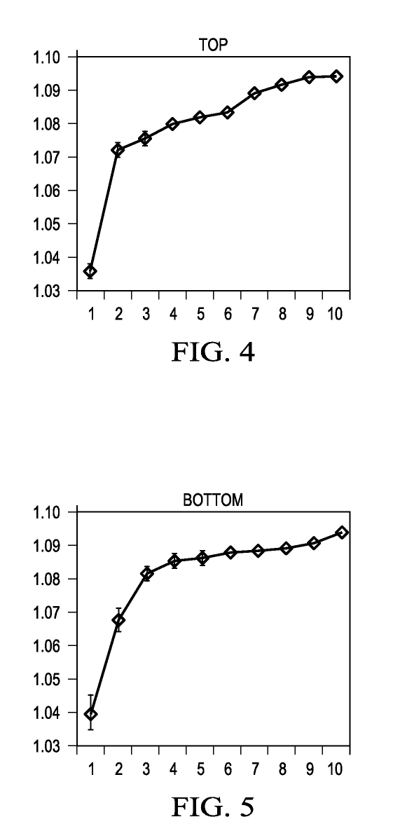

FIGS. 4 and 5 are graphs that show that the continuous density gradient can be maintained in both top and bottom loading methods during the purification.

After centrifugation, the gradient solution was collected into 10 tubes from the top to the bottom. In COBE method, about 80 ml of the solution was left in the COBE bag structurally. On the other hand, whole solution was collected in the bottle method, which prevents the loss of islets. Samples were taken from each tube, stained with dithizone and assessed for islet purity. Islet-rich tubes were selected and the tissues were collected.

Islet Evaluation. Islet preparations were evaluated for yield, purity and morphology by using dithizone (3 mg/ml) (Sigma Chemical Co., St. Louis, Mo.) staining. The crude number of islets in each diameter class was determined by counting islets using an optical graticule. The crude number of islets was then converted to the standard number of islet equivalents (IE; diameter standardizing to 150 μm). Gross morphology was qualitatively assessed by scoring the islets for shape (flat vs. spherical), border (irregular vs. well-rounded), integrity (fragmented vs. solid/compact), uniformity of staining (not uniform vs. perfectly uniform) and diameter (least desirable: all cells<100 μm/most desirable: more than 10% of the cells>200 μm). Each parameter was graded from zero (the worst) to two (the best), so that the worst islet preparations were given a cumulative score of zero and the best a score of ten. Spherical, well-rounded, solid/compact, uniformly stained and large islets were characterized as the best islets. FIG. 7 are images showing the morphology of Post-purification Islets. The same volume samples after purification were taken from three methods

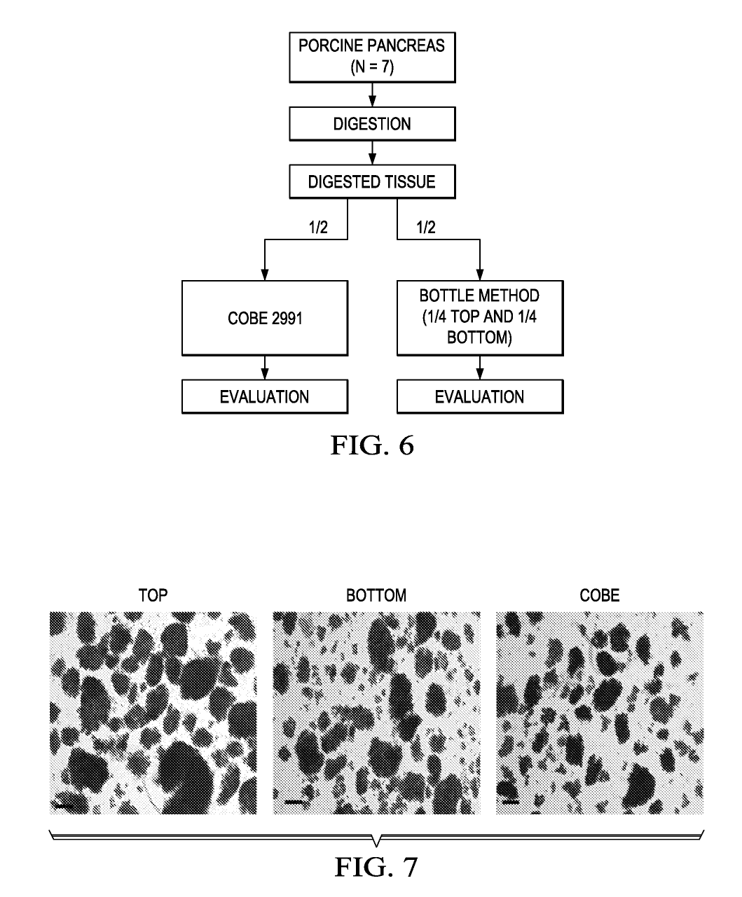

FIG. 6 shows the steps followed to compare the current art with the present invention. Islet viability after purification was assessed using trypan blue staining. Fifty islets were inspected and their individual viability was determined visually, followed by calculation of their average viability.

In vitro islet function was assessed by monitoring the insulin secretory response of the islets during glucose stimulation as described before⁸. Briefly, 5,000 IE were incubated with either 2.8 mM or 25 mM glucose in RPMI 1640 for 2 hours at 37° C. in a 5% CO2 atmosphere. The supernatant was collected and insulin levels were determined using an enzyme-linked immunosorbent assay kit (ALPCO Diagnostics, Windham, N.H.). The stimulation index was calculated by determining the ratio of insulin released from islets in the high glucose concentration to the low concentration. The data were normalized by total DNA.

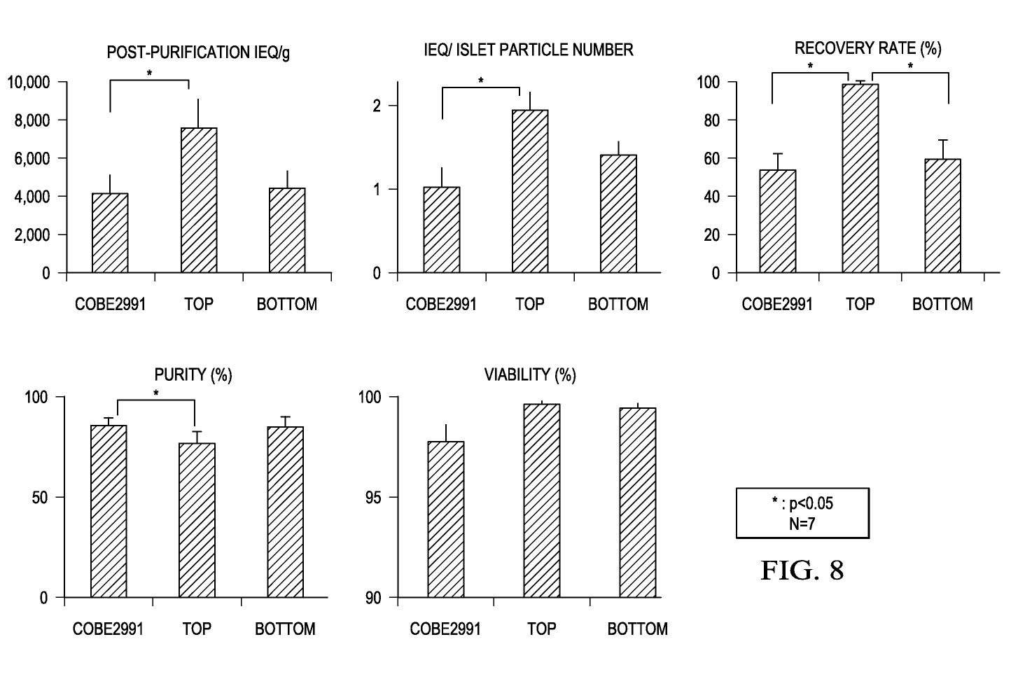

FIG. 8 shows several graphs that compare the results of the prior art (COBE2991) versus the top loading and bottom loading method using bottle purification of Pancreatic Islets for: Post-purification IEQs, IEQ to Islet Particle No.; Recovery rate (%); Purity (%); and Viability (%).

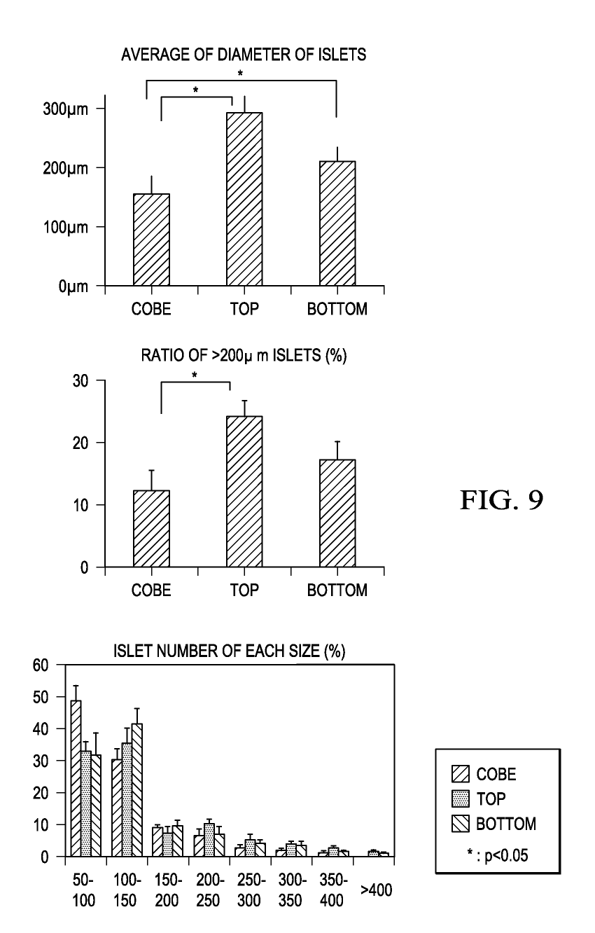

FIG. 9 includes graphs that compare the Pancreatic Islet size for: Average Islet Diameter; Ratio of Islets>200 μm; and Average Islet Size Distribution (%).

In vivo assessment of islet function. Nude mice (Harlan, Houston, Tex.) rendered diabetic by a single injection of streptozotocin (STZ) at a dose of 120 mg/kg were used for the study. When the non-fasting blood glucose level exceeded 350 mg/dl for two consecutive days, the mouse was considered to be diabetic. The 10,000 IE pig islets obtained from each group were transplanted into the renal subcapsular space of the left kidney of a diabetic nude mouse immediately after the isolation. During the thirty-day posttransplantation period, the non-fasting blood glucose levels were measured at three times per week. Normoglycemia was defined when two consecutive blood glucose level measurements showed less than 200 mg/dl. No statistical differences in either pretransplantation blood glucose levels or pretransplantation body weight were observed among the three groups. These studies were approved by the Institutional Animal Care and Use Committee (IACUC) of Baylor Health Care System.

Statistical Analysis: All results were expressed as the means±SE. Differences among 3 groups were analyzed by ANOVA followed by Student's t-test with Bonferroni correction. P-values less than 0.05 were considered significant in ANOVA and in Student's t-test with Bonferroni correction. Differences of ratio among 3 groups were analyzed by Chi-squared test with Ryan method.

Stability of the density gradient in the bottle purification methods: After centrifugation, the solution was collected into 10 tubes. We measured the density of each tube, and confirmed that the continuous density gradients were maintained during the whole purification procedure in both Top and Bottom methods (FIGS. 4 and 5).

A total of ten porcine pancreata were used in the present study. The islet isolation variables are shown in Table 2. The average of the weight of used pancreata was 136±17 g. The average of the warm ischemic time was 46±2 minutes, therefore, these pancreata were in a marginal condition.

TABLE 2
Isolation Variables of Porcine Pancreas

| | |
|---|---|
| Age (Year) | 2 |
| Warm Ischemic Time (min) | 46 ± 2 |
| Cold Ischemic Time (min) | 121 ± 1 |
| Phase1 (min) | 13 ± 1 |
| Phase2 (min) | 41 ± 2 |
| Pancreas Weight (g) | 136 ± 17 |
| Pre-purification IEQ | 988,016 ± 107,491 |
| Pre-purification IEQ/g | 7,933 ± 919 |
| Digested Tissue Volume (ml) | 26 ± 4 |
| Undigested Tissue (g) | 24 ± 5 |

Data are expressed as mean ± SE.

Post-purification variables and the results are shown in Tables 3 and 4, respectively. Islet yield (IE) per pancreas weight (IE/g) after purification was significantly higher in the Top group than COBE group (IE/g=Top: 8060±1652 IE/g, Bottom: 4572±614 IE/g, COBE: 3900±734 IE/g. p<0.02 in Top vs. COBE). The rate of post-purification recovery were significantly higher in the Top group than both Bottom and COBE groups (percentage of recovery=Top: 99.3±12.3%, Bottom: 62.6±8.8%, COBE: 49.5±6.7%. p<0.02 in Top vs. Bottom and p<0.001 in Top vs. COBE, respectively).

TABLE 3
Post-purification variables.

| | | | | Average | |
|---|---|---|---|---|---|
| | Purity | Viability | Recovery | size of | Stimulation |
| | (%) | (%) | rate (%) | islets (μm) | Index |
| Top | 62.2 ± 7.9 | 98.7 ± 0.8 | 99.3 ± 12.3 | 156 ± 8 | 5.4 ± 1.9 |
| Bottom | 73.7 ± 5.7 | 99.2 ± 0.2 | 62.6 ± 8.8 | 147 ± 6 | 7.2 ± 5.1 |
| COBE | 68.8 ± 7.4 | 97.7 ± 0.7 | 49.5 ± 6.7 | 119 ± 6 | 3.1 ± 1.4 |
| P value | 0.211 | 0.311 | 0.001 | 0.004 | 0.390 |
| Top vs. | | | | | |
| COBE | | | | | |
| p value | 0.009 | 0.422 | 0.017 | 0.145 | 0.757 |
| Top vs. | | | | | |
| Bottom | | | | | |

Data are expressed as mean ± SE.
Recovery rate (%): IE after purification/IE before purification × 100

TABLE 4
Post Purification Results of 3 methods

| | | IEQ/Islet Particle | | | Recovery |
|---|---|---|---|---|---|
| | IE/g | Number | Purity(%) | Viability(%) | rate(%) |
| COBE 2991 | 4114 ± 953 | 1.03 ± 0.2 | 86.0 ± 3.8 | 97.7 ± 0.9 | 53.4 ± 9.3 |
| Top | 7557 ± 1528 | 1.94 ± 0.2 | 76.9 ± 5.5 | 99.6 ± 0.1 | 98.4 ± 14.7 |
| Bottom | 4422 ± 857 | 1.40 ± 0.2 | 85.1 ± 5.2 | 99.4 ± 0.2 | 59.5 ± 9.8 |
| p value Top | 0.029 | 0.003 | 0.021 | 0.065 | 0.012 |
| vs. COBE | | | | | |
| p value Top | 0.052 | 0.055 | 0.026 | 0.161 | 0.026 |
| vs. Bottom | | | | | |

Top loading method resulted in significantly higher IEQ/g, IEQ/islet particle number and recovery rate compared to both COBE 2991 and bottom loading method.

Top loading method (Top) resulted in significantly higher IEQ/g, IEQ/islet particle number and recovery rate compared to both COBE 2991 and bottom loading method. The islet size in Top group was significantly larger than COBE, which indicates that Top-loading method caused less shearing stress on islets during the purification procedure.

Top-loading also gave rise to better results than bottom loading. Some islets might be captured by acinar cells and be not able to ascend to the low density layer during bottom loading method, thereby reducing yield. The top loading method prevented the islets from falling apart and improved the post purification IEQ.

The size of isolated islets after purification: The average diameter of purified islets and the rate of more than 200 μm sized islets were significantly higher in the Top and Bottom group than COBE group (Average of the diameter of islets=Top: 156±8 μm, Bottom: 147±6 μm, COBE: 119±6 μm. p<0.01 in Top vs. COBE and in Bottom vs. COBE; Percentage of >200 μm size islets=Top: 26.0±3.2%, Bottom: 20.0±3.0%, COBE: 10.8±2.6%. p<0.02 in Top vs. COBE and in Bottom vs. COBE).

Assessment of islet function in vitro and in vivo: Stimulation index in the Bottom group was higher than the other 2 groups, although there was no significant difference (Top: 5.4±1.9, Bottom: 7.2±5.1, COBE: 3.1±1.4, respectively, Table 3).

To assess the islet graft function in vivo, 10,000 IE islets of each group were transplanted under the kidney capsule of STZ-induced diabetic nude mice. The blood glucose levels in 7 of 11 mice (64%) in Top group, 5 of 11 mice (45%) in Bottom group and 2 of 5 mice (40%) in COBE group decreased and reached normoglycemia (Tables 5 and 6). The ratio of the cured mice in Top group was the highest among 3 groups, although there was no statistical significance.

In summary, the purification with Top loading method resulted in the highest yield and the largest size of islets. These data suggest that Top loading was the best purification method for porcine islet isolation among three groups.

TABLE 5
In vivo Functional Assay

| | n | % cured mice |
|---|---|---|
| COBE | 8 | 0 |
| Top | 7 | 14.3 |
| Bottom | 8 | 25.0 |

TABLE 6
In vivo Functional Assay

| | Transplanted mice | Cured mice | % | p value vs. COBE |
|---|---|---|---|---|
| Top | 11 | 7 | 64 | NS |
| Bottom | 11 | 5 | 45 | NS |
| COBE | 5 | 2 | 40 | |

The 10,000 IEQ of the fresh islets were transplanted into diabetic nude mice. The normoglycemia was defined as <200 mg/dl in non-fasting condition in more than two consecutive days.
NS: not significant

Islet allotransplantation is a promising therapeutic method for diabetes. However, this relatively safe and minimally invasive therapy still faces a difficult problem of a limited supply of human pancreas donors. Porcine islet cells are a good substitute for human islet cells. However, porcine islets are known to be difficult to isolate because of a lack of a surrounding islet capsule¹² and the fragility to be easily fragmented¹³ during isolation.

The present invention demonstrates a purification method using large bottles that improves the islet isolation outcome for porcine pancreas¹⁴. In the present study, the bottom loading method and discontinuous density solution were used for the purification. The continuous density gradient of the present invention separates islets from other cells more efficiently than discontinuous solution. Therefore, the present study is not only the first report of a direct comparison of porcine islet purification methods between COBE and large bottles, but also a direct comparison between Top loading and bottom loading method with continuous density gradient.

Another modification of the present invention is that the digested tissue was mixed with low density solution instead of preservation solution such as University of Wisconsin (UW) solution, which is the standard method in the case of COBE. The present inventors in the Top loading method, mixed the digest in UW solution and loaded it to the upper layer, which made a wall of tissue between the density gradient solution and the tissue solution because the density of UW solution is much lower (1.040 g/ml) than light solution (1.077 g/ml). The tissue was precipitated at the bottom of UW solution before the centrifugation and this condensed tissue causing a lot of islets to be entrapped with the acinar tissue and dragged into the bottom. This should not occur in COBE, since the COBE bag is centrifuged during the tissue loading. On the other hand, when the tissue was mixed with the low density solution, there was not such a wall because we used the same density of solution for the tissue and light solution. Then the relatively heavy acinar tissues started to move to the bottom of the bottle soon after they were loaded, before the centrifugation, minimizing the dragged islets.

With Top loading method, the inventors isolated large quantities of islets from porcine pancreata. Several factors may be considered to be important. First, bottle methods can minimize the shearing force during the centrifugation for the purification. As shown previously, COBE bag has narrow segment and islets suffered significant shearing force during passage through this narrow segment. Since the bottle does not have such narrow segment, the shear stress could be substantially reduced. In fact, this should be the reason why the size of purified islets in both Top and Bottom method was significantly larger than COBE. Since porcine islets are fragile, to minimize the mechanical damage during the isolation must be critical.

Second, the pancreata used in this study were in a marginal condition. The average of the warm ischemic time was more than 45 minutes, due to the speed of dressing the pigs at a local slaughterhouse. Even 30 minutes warm ischemic condition can cause severe apoptosis in porcine islets¹⁵, therefore islets in this study are considered to be significantly damaged. Indeed, for the in vivo functional analysis, 10,000 IE islets were required to cure diabetic mice. To put it differently, however, the modified isolation method of the present invention enables the obtaining of functional islets from porcine pancreata under such severely adverse conditions. Although there was differences did not reach statistical significant, the islets in Top group had the highest quality according to the in vitro and in vivo evaluation.

Third, Top loading has several advantages over Bottom loading. In the case of Bottom loading, the purity of islets was the highest among 3 groups but IE/g was less than Top loading. The present inventors assume that it is partially because many islets could be entrapped by the vicinal acinar tissue during making the density gradient solution, and they could not float up to the upper layer. In addition, the relatively longer time when the loaded islets were exposed to the high density solution during Bottom loading might damage the islets, whereas islets were exposed to the low density solution in Top loading.

Moreover, the bottle purification method of the present invention is simple and easy to perform. It eliminates the cost and experience of COBE2991 cell processor, and it may contribute to the spread of islet isolation. The newly developed purification method using large bottles with a top loading as described in the present invention enables obtaining an extremely high yield of porcine islets.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 7,045,349: Method of islet isolation using process control. United States Patent Application No. 20080038823: Method of Separating Pancreatic Islet.

1. Shintaku, H.; Okitsu, T.; Kawano, S.; Matsumoto, S.; Suzuki, T.; Kanno, I.; Kotera, H. Effects of fluid dynamic stress on fracturing of cell-aggregated tissue during purification for islets of Langerhans transplantation. J. Physics D: Applied Physics. 41: 115507 (9 pp); 2008.

2. Noguchi, H.; Ueda, M.; Hayashi, S.; Kobayashi, N.; Nagata, H.; Iwanaga, Y.; Okitsu, T.; Matsumoto, S. Comparison of M-Kyoto solution and histidine-tryptophan-ketoglutarate solution with a trypsin inhibitor for pancreas preservation in islet transplantation. Transplantation. 84:655; 2007.

3. Noguchi, H.; Ueda, M.; Nakai, Y.; Iwanaga, Y.; Okitsu, T.; Nagata, H.; Yonekawa, Y.; Kobayashi, N.; Nakamura, T.; Wada, H.; Matsumoto, S. Modified two-layer preservation method (M-Kyoto/PFC) improves islet yields in islet isolation. Am. J. Transplant. 6:496; 2006.

4. Matsumoto, S.; Okitsu, T.; Iwanaga, Y.; Noguchi, H.; Nagata, H.; Yonekawa, Y.; Yamada, Y.; Fukuda, K.; Tsukiyama, K.; Suzuki, H.; Kawasaki, Y.; Shimodaira, M.; Matsuoka, K.; Shibata, T.; Kasai, Y.; Maekawa, T.; Shapiro, J.; Tanaka K. Insulin independence after living-donor distal pancreatectomy and islet allotransplantation. Lancet. 365:1642-1644; 2005.

5. Matsumoto, S.; Qualley, S. A.; Goel, S.; Hagman, D. K.; Sweet, I. R.; Poitout, V.; Strong, D. M.; Robertson, R. P.; Reems, J. A. Effect of the two-layer (University of Wisconsin solution-perfluorochemical plus O2) method of pancreas preservation on human islet isolation, as assessed by the Edmonton Isolation Protocol. Transplantation. 74:1414-1419; 2002.

6. Ricordi, C. Islet transplantation: a brave new world. Diabetes. 52:1595-1603; 2003.

7. Ricordi, C.; Finke, E. H.; Dye, E. S.; Socci, C.; Lacy, P. E. Automated method for isolation of human pancreatic islets. Diabetes. 37: 413-420; 1988.

8. Shapiro, A. M.; Lakey, J. R.; Ryan, E. A.; Korbutt, G. S.; Toth, E.; Warnock, G. L.; Kneteman, N. M.; Rajotte, R. V. Islet transplantation in seven patients with type 1 diabetes mellitus using a glucocorticoid-free immunosuppressive regimen. N. Engl. J. Med. 343:230-238; 2000.

9. Matsumoto, S.; Shibata, S.; Kirchihof, N.; Hiraoka, K.; Sageshima, J.; Zhang, X.; Gilmore, T.; Ansite, J.; Zhang, H.; Sutherland, D. E. R.; Hering, B. J. Immediate reversal of diabetes in primates following intraportal transplantation of porcine islets purified on a new histidine-lactobionate-iodixanol gradient. Transplantation. 67:S220; 1999.

10. Matsumoto, S.; Zhang, H. J.; Gilmore, T.; Van der Burg, M. P.; Sutherland, D. E. R.; Hering, B. J. Large-scale isopycnic islet purification utilizing non-toxic, endotoxin-free media facilitates immediate single-donor pig islet allograft function. Transplantation 66:S30; 1998.

11. Noguchi, H.; Ikemoto, T.; Naziruddin, B.; Jackson, A.; Shimoda, M.; Fujita, Y.; Chujo, D.; Takita, M.; Kobayashi, N.; Onaca, N.; Levy, M. F.; Matsumoto, S. Iodixanol-controlled density gradient during islet purification improves recovery rate in human islet isolation. Transplantation. 87:1629-35; 2009.

12. Meyer, T.; Czub, S.; Chodnewska, I.; Beutner, U.; Hamelmann, W.; Klöck, G.; Zimmermann, U.; Thiede, A.; Ulrichs, K. Expression pattern of extracellular matrix proteins in the pancreas of various domestic pig breeds, the Goettingen Minipig and the Wild Boar Ann Transplant 2:17-26; 1997.

13. Swanson, C. J.; Olack, B. J.; Goodnight, D.; Zhang, L.; Mohanakumar, T. Improved methods for the isolation and purification of porcine islets. Hum. Immunol. 62:739-749; 2001.

14. Yonekawa, Y.; Matsumoto, S.; Okitsu, T.; Arata, T.; Iwanaga, Y.; Noguchi, H.; Nagata, H.; O'Neil, J. J.; Tanaka, K. Effective islet isolation method with extremely high islet yields from adult pigs. Cell Transplant. 14:757-62; 2005.

15. Troisi, R.; Meester, D.; Van Den Broecke, C.; Cuvelier, C. A.; Fiers, T.; de Hemptinne, B.; Hesse, U. J. Functional and structural integrity of porcine pancreatic grafts subjected to a period of warm ischemia and cold preservation with histidine-tryptophan-ketoglutarate (custodiol) or University of Wisconsin solution. Transplantation. 75:1793-9; 2003.

What is claimed is:

1. A method of isolating pancreatic islets comprising:
creating a continuous density gradient in a vessel comprising at least 100 milliliters with a bend tube that reduces convection currents that disrupt the gradient;
loading a solution comprising pancreatic islets in the density gradient;
centrifuging the vessel wherein the pressure on the pancreatic islets is less than 50 Pa; and
isolating the pancreatic islets from the gradient.

2. The method of claim 1, wherein the gradient and the solution comprising pancreatic islets are topped with a dilution solution.

3. The method of claim 1, wherein the pancreatic islets are top-loaded and in which the gradient comprises:
a high-density first layer at the bottom of the vessel,
a second layer comprising the continuous density gradient on the high density first layer,
a third layer on the second layer, the third layer comprising a low-density solution that comprises a pancreatic digest; and
a fourth layer on the third layer comprising a dilution solution.

4. The method of claim 1, wherein the pancreatic islets are bottom-loaded and in which the gradient comprises:
a high-density first layer comprising a pancreatic digest at the bottom of the vessel; a second layer on the first layer comprising a continuous density gradient; and
a third layer comprising a dilution solution.

5. The method of claim 1, wherein the gradient comprises a continuous density solution, further defined as comprising a low-density to a high-density by changing the volumetric ratio of low-density to high-density using the bend tube.

6. The method of claim 1, wherein the gradient comprises a continuous density solution, further defined as comprising a low-density to a high-density comprising Iodixanol to ET-Kyoto solutions produced by changing the volumetric ratio of Iodixanol and ET-Kyoto solution using the bend tube.

7. The method of claim 1, wherein the gradient comprises a continuous density solution, further defined as comprising a low-density (density: 1.077) to a high-density (density: 1.095-1.125) Iodixanol-ET-Kyoto solutions produced by changing the volumetric ratio of Iodixanol and ET-Kyoto solution using the bend tube.

8. The method of claim 1, wherein the bottle has a smooth inner surface.

9. The method of claim 1, wherein the gradient is formed using a bent-tip that reduces convection currents in the gradient when the gradient is created.

10. The method of claim 1, wherein the vessel is 200, 250, 300, 400, 500, 600, 700, 800, 900 or 1,000 milliliters.

11. The method of claim 1, further comprising the step of isolating the islet cells from the collection vessel comprising the islet cells.

12. The method of claim 1, wherein the further comprising the steps of:
isolating the islet cells from the collection vessel comprising the islet cells;
washing the islet cells; and
transplanting the pancreatic islets into a new host.

13. The method of claim 1, wherein the islets are human islets.

14. The method of claim 1, wherein the pancreatic islets are cadaveric islets.

15. A method of isolating pancreatic islets comprising:
creating a continuous density gradient in a vessel comprising at least 100 milliliters with a bend tube that reduces convection currents that disrupt the gradient, the continuous density solution, further defined as comprising a low-density (density: 1.077) to a high-density (density: 1.095-1.125) prepared using a bend tube;
loading pancreatic islet cells in a dilution solution on the gradient;
centrifuging the vessel wherein the pressure on the pancreatic islets is less than 50 Pa;
separating into two or more collection vessels the gradient from the top to the bottom of the vessel;
selecting the collection vessel with the pancreatic islets; and
isolating the pancreatic islets from the gradient.

16. The method of claim 15, wherein the gradient and the solution comprising pancreatic islets are topped with a dilution solution.

17. The method of claim 15, wherein the pancreatic islets are top-loaded and in which the gradient comprises:
a high-density first layer at the bottom of the vessel,
a second layer comprising the continuous density gradient on the high density first layer,
a third layer on the second layer, the third layer comprising a low-density solution that comprises a pancreatic digest; and
a fourth layer on the third layer comprising a dilution solution.

18. The method of claim 15, wherein the pancreatic islets are bottom-loaded and in which the gradient comprises:
a high-density first layer comprising a pancreatic digest at the bottom of the vessel; a second layer on the first layer comprising a continuous density gradient; and
a third layer comprising a dilution solution.

19. The method of claim 15, wherein the gradient comprises a continuous density solution, further defined as comprising a low-density (density: 1.077) to a high-density (density: 1.095-1.125) Iodixanol-ET-Kyoto solutions produced by changing the volumetric ratio of Iodixanol and ET-Kyoto solution.

20. The method of claim 15, wherein the vessel is 150, 200, 250, 300, 400, 500, 600, 700, 800, 900 or 1,000 milliliters.

21. The method of claim 15, wherein the further comprising transplanting the pancreatic islets into a new host.

22. The method of claim 15, wherein the islets are human islets.

23. The method of claim 15, wherein the pancreatic islets are cadaveric islets.

24. A method of isolating pancreatic islets comprising:
creating a density gradient in a vessel comprising at least 100 milliliters;
loading a solution comprising pancreatic islets in a dilution solution below the density gradient;
centrifuging the vessel wherein the pressure on the pancreatic islets is less than 50 Pa;
separating into two or more collection vessels the gradient from the top to the bottom of the vessel;
selecting the collection vessel with the pancreatic islets; and
isolating the pancreatic islets from the gradient.

25. A method of isolating pancreatic islets by density centrifugation wherein the pancreatic islets are loaded in a solution comprising pancreatic islets with the density gradient and the islets are isolated by centrifuging the vessel wherein the pressure on the pancreatic islets is less than 50 Pa and wherein the pancreatic islets are isolated from the gradient, the improvement comprising creating a continuous density gradient in a vessel comprising at least 100 milliliters with a bend tube that reduces convection currents that disrupt the gradient.

26. One or more pancreatic cells isolated by the method comprising:
creating a continuous density gradient in a vessel comprising at least 100 milliliters with a bend tube that reduces convection currents that disrupt the gradient;
loading a solution comprising pancreatic islets in the density gradient;
centrifuging the vessel wherein the pressure on the pancreatic islets is less than 50 Pa; and
isolating the pancreatic islets from the gradient.

27. The cells of claim 26, wherein the gradient and the solution comprising pancreatic islets are topped with a dilution solution.

28. The cells of claim 26, wherein the pancreatic islets are top-loaded and in which the gradient comprises:
a high-density first layer at the bottom of the vessel,
a second layer comprising the continuous density gradient on the high density first layer,
a third layer on the second layer, the third layer comprising a low-density solution that comprises a pancreatic digest; and
a fourth layer on the third layer comprising a dilution solution.

29. The cells of claim 26, wherein the pancreatic islets are bottom-loaded and in which the gradient comprises:
a high-density first layer comprising a pancreatic digest at the bottom of the vessel; a second layer on the first layer comprising a continuous density gradient; and
a third layer comprising a dilution solution.

30. The cells of claim 26, wherein the gradient comprises a continuous density solution, further defined as comprising a low-density to a high-density by changing the volumetric ratio of low-density to high-density using the bend tube.

31. The cells of claim 26, wherein the gradient comprises a continuous density solution, further defined as comprising a low-density to a high-density comprising Iodixanol to ET-Kyoto solutions produced by changing the volumetric ratio of Iodixanol and ET-Kyoto solution using the bend tube.

32. The cells of claim 26, wherein the gradient comprises a continuous density solution, further defined as comprising a low-density (density: 1.077) to a high-density (density: 1.095-1.125) Iodixanol-ET-Kyoto solutions produced by changing the volumetric ratio of Iodixanol and ET-Kyoto solution using the bend tube.
